(19) United States
(12) Patent Application Publication
(10) Pub. No.: US 2016/0303283 A1
(43) Pub. Date: Oct. 20, 2016

(54) THIN FILM WITH MICROCHANNELS

(71) Applicant: Worcester Polytechnic Institute, Worcester, MA (US)

(72) Inventors: Keith A. Gagnon, Worcester, MA (US); Thomas J. Moutinho, JR., Worcester, MA (US); Kevin A. Reyer, Worcester, MA (US); Alyssa L. Bornstein, Worcester, MA (US); Megan O'Brien, Worcester, MA (US); George D. Pins, Worcester, MA (US); Glenn R. Gaudette, Worcester, MA (US)

(21) Appl. No.: 15/099,957

(22) Filed: Apr. 15, 2016

Related U.S. Application Data
(60) Provisional application No. 62/149,168, filed on Apr. 17, 2015.

(51) Int. Cl. A61L 27/22; A61L 27/54; A61L 27/38; A61L 27/52; B29C 33/38; G03F 7/32; B29C 41/00; B29C 41/42; B29C 65/00; B29C 41/20; G03F 7/20; G03F 7/38; A61K 35/34; B29C 33/40

(52) U.S. Cl. CPC A61L 27/225; A61K 35/34; A61L 27/54; A61L 27/3826; A61L 27/52; B29C 33/3842; B29C 33/40; B29C 41/003; B29C 41/42; B29C 65/002; B29C 41/20; G03F 7/2002; G03F 7/38; G03F 7/32; B29K 2883/00; B29L 2031/753

(57) ABSTRACT

A scaffold, a method of using the scaffold and a method of preparing the scaffold which may promote the growth or survival of cells and tissues. The scaffold comprises at least one microvascular layer formed from a thin-film fibrin, the microvascular layer configured to sustain and promote growth of cells and having one or more microfluidics channels embedded in the microvascular layer, the channels configured to contain nutrients needed for growth of the cells, and the channels configured to permit diffusion of the nutrients from the channels to the cells.

### CROSS-REFERENCE TO RELATED APPLICATIONS

This application claims priority to U.S. Provisional Patent Application Ser. 62/149,168, filed. Apr. 17, 2015, the entirety of which is incorporated herein by reference.

### STATEMENT OF GOVERNMENT SUPPORT

This invention was made with Government Support under Contract Number HL 115282 awarded by the National Institute of Health. The Government has certain rights in the invention.

### FIELD

The present disclosure relates to thin film hydrogels with an engineered microvascular network.

### BACKGROUND

A major challenge in cardiac tissue engineering is the perfusion and adequate delivery of oxygen and nutrients to metabolically active cells throughout an engineered construct. Growing large tissue constructs without some mechanism of implanted vascularization will lead to tissue necrosis in the center of the engineered construct, caused by excessive distance between the cells and the nutrient source.

Previous attempts at engineered vasculature have resulted in large, unperfused gaps in the tissue due to the lack of control over the orientation, size and spacing of the vessels. There is still a need for a vascularized thin film for use in tissue engineering scaffolds.

### SUMMARY

Some embodiments of the present disclosure provide a thin film hydrogel having an engineered microvascular network capable of supporting continuous perfusion, which is modular, maneuverable and able to be stacked as part of a layer-by-layer construct. The microvascular network is able to be perfused in an un-endothelialized state, allowing for a reduced time from synthesis to implantation, as growth of endothelialized vessels are not needed to contain flow. In some embodiments, there are provided modular, multi-layered scaffolds including thin films of the present disclosure. In some embodiments, such scaffolds can be used for the creating of cardiac tissue. In some embodiments, the scaffolds may be manufactured in a layer-by-layer construction in which 200 μm thick vascular layers are stacked with 200 μm thick functional tissue layers, thus ensuring that no point in the functional tissue layer exists which is greater than 200 μm from a vascular source.

In some embodiments, in accordance with the present disclosure, there is provided a scaffold comprising at least one microvascular layer formed from a thin-film fibrin, the microvascular layer configured to sustain and promote growth of cells and having one or more microfluidics channels embedded in the microvascular layer, the channels configured to contain nutrients needed for growth of the cells, and the channels configured to permit diffusion of the nutrients from the channels to the cells. In some embodiments, the microvascular layer comprises a thickness of from about 100 microns to about 600 microns. In some embodiments, the microvascular layer comprises a thickness of from about 200 microns to about 400 microns. In some embodiments, the cells are disposed in the microvascular layer. In some embodiments, the cells are disposed in the microvascular layer at a distance of no greater than 200 microns from the microfluidics channels. In some embodiments, the cells are stem cells. In some embodiments, the cells are cardiac cells.

In some embodiments, the scaffold comprises multiple microvascular layers, the layers being stacked on top of each other. In some embodiments, the scaffold further comprises at least one functional tissue layer having cells disposed within the functional layer, the microvascular layer being configured to permit diffusion of the nutrients from the microvascular layer to the cells in the functional layer. In some embodiments, the scaffold comprises multiple functional layers and multiple microvascular layers, the layers being stacked and arranged such that at least one functional layer is positioned on each side of a microvascular layer.

In some embodiments, the functional layer and the microvascular layer comprise a thickness of from about 100 microns to about 200 microns. In some embodiments, the cells are disposed in the functional layer at a distance of no greater than 200 microns from the microfluidics channels in the microvascular layer.

In some embodiments, the microfluidics channels are square or rectangular shaped channels. In some embodiments, the microfluidics channels comprise a width of from 10 to 800 microns, and a height of from 10 to 190 microns. In some embodiments, the microfluidics channels are circular shaped channels. In some embodiments the microfluidics channels comprise a branching structure. In some embodiments, the branching structure is square branching, circular branching or triangular branching.

In another embodiment, a method for sustaining cell survival in an individual comprising providing a scaffold, inoculating the microvascular layer with the cells, and positioning the scaffold at a desired location within the individual. In some embodiments, the scaffold comprises at least one microvascular layer formed from a thin-film fibrin and configured to sustain and promote growth of cells, the microvascular layer having one or more microfluidics channels embedded in the microvascular layer, the channels configured to contain nutrients needed for growth of the cells, and the channels configured to permit diffusion of the nutrients from the channels to the cells. In some embodiments, the scaffold comprises more than one microvascular layer, the scaffold being constructed in a layer by layer manner to achieve a layered scaffold of a desired thickness.

In some embodiments, the scaffold further comprises a functional tissue layer having cells disposed within the functional layer, the microvascular layer being configured to permit diffusion of the nutrients from the microvascular layer to the functional layer, the microvascular and functional layers defining a perfused tissue scaffold.

In some embodiments, the individual comprises an individual who has sufferered ischemic or reperfusion damage to a tissue. In some embodiments, the damage comprises damage to an organ, wherein the organ may be a kidney, a heart, a brain, a liver, or a lung.

In another embodiment, a method of manufacturing a thin-film fibrin scaffold comprising filling a first scaffold mold with a fibrin hydrogel, cross-linking the fibrin hydrogel to yield a first half layer of a thin-film fibrin scaffold, concomitantly to filling the first mold, filling a second scaffold mold with the fibrin hydrogel, cross-linking the fibrin hydrogel to yield a second half layer of the thin-film fibrin scaffold, and combining the two half layers before the cross-linking is complete to yield a thin-film fibrin scaffold. The scaffold comprises one or more microfluidics channels, the channels configured to contain nutrients needed for growth of cells and configured to permit diffusion of the nutrients from the channels to the cells. In some embodiments, the hydrogel may have a concentration of from 10 to 30 mg/ml fibrin.

In some embodiments, the method further comprises, before the step of filling the first scaffold mold, the step of patterning a mask for a scaffold mold on to a negative photoresist substrate to yield a patterned substrate, and pouring an organic compound into the patterned substrate and allowing the organic compound to harden, yielding a scaffold mold. In some embodiments, the organic compound is polydimethylsiloxane (PDMS). In some embodiments, the patterning comprises photolithography.

### DESCRIPTION OF THE DRAWINGS

The presently disclosed embodiments will be further explained with reference to the attached drawings. The drawings shown are not necessarily to scale, with emphasis instead generally being placed upon illustrating the principles of the presently disclosed embodiments.

FIG. 1 is an embodiment of a microvascular channel network in accordance with the present disclosure.

FIGS. 2A and 2B illustrate various designs for a microvascular network for an thin-film, endothelialized microvascular layer in accordance with the present disclosure.

FIG. 3A illustrates an embodiment of a scaffold having multiple microvascular layers in accordance with the present disclosure.

FIG. 3B illustrates an embodiment of a scaffold with multiple microvascular layers and functional tissue layers in accordance with the present disclosure.

FIG. 3C depicts an embodiment of a scaffold for regenerating cardiac tissue in accordance with the present disclosure.

FIG. 4A depicts a schematic of a design of a fibrin-based microfluidic network in accordance with the present disclosure.

FIG. 4B is a picture of an embodiment of a silicon wafer in accordance with the present disclosure.

FIGS. 5A and 5B depict microchannel branching dimensions in a polydimethylsiloxane (PDMS) mold (5A) and a fibrin (5B) gel cast from that mold.

FIG. 5C illustrates a quantitative comparison of microchannel widths in a fibrin gel and a PDMS mold.

FIG. 6A illustrates an embodiment of a discretely loaded fibrin microvascular network perfused with blue microbeads in accordance with the present disclosure.

FIG. 6B illustrates an embodiment of a layered fibrin perfusion with blue microbeads loaded into a gel in accordance with the present disclosure.

FIG. 7 depicts an embodiment of a single channel fibrin hydrogel in accordance with the present disclosure.

FIGS. 8A-C depicts embodiments of three junction geometries for microchannel networks, square (8A), circular (8B) and triangular branching (8C).

FIGS. 9A-C depicts an embodiment of a width profile analysis of the vascular network, showing constant width (9A), stepping width (9B) and Murray's Law (9C).

FIG. 10 illustrates an embodiment of a microfluidic network in accordance with the present disclosure.

FIG. 11 graphs a flow and a diffusion of Fluorescein isothiocyanate (FITC) in a single channel fibrin channel.

FIG. 12A is a chart of intensity versus change in distance from channel via a fluorescence profile.

In FIG. 12B is a linear regression graph showing the relationship between the Full Width at Half Maximum (FWHM) of the intensity profile and time since the initiation of perfusion.

FIG. 13 illustrates a cellular viability testing apparatus in accordance with the present disclosure.

FIGS. 14A-F present fluorescent microscopy images of experimental test results for embodiments of endothelialized layers thin film in accordance with the present disclosure.

FIGS. 14A-14D are images from gels perfused with media, while FIGS. 14E and 14F are images from a gel perfused with PBS.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

In some embodiments, the present disclosure provides a thin film fibrin-based scaffold which may contain a physiologically relevant, microfluidics-based, branched microvascular network (also referred to herein as microchannels) configured to support cells by supplying nutrients or a nutrient source to the cells. In some embodiments, a layer-by-layer construction scaffold is provided in which a microvascular layer is generated which may contain a microchannel network and may further comprise cells. The microvascular layer may be a thickness of from about 100 μm (microns) to about 600 μm, of from about 150 μm to about 500 μm, of from about 150 μm to about 400 μm, of from about 150 μm to about 300 μm, or may be about 200 μm. The microvascular layer may contain more than one set of microchannels. The microvascular layers may be stacked with functional tissue layers. Each layer may be of the same thickness or may be different thickness. The functional tissue layer may be a thickness of about 200 μm. In some embodiments, the functional layer may be of a thickness that ensures that no point in the functional tissue layer exists which is greater than 200 μm from a vascular source. In some embodiments, a scaffold for tissue engineering is provided where the cells are between 150 μm and 200 μm from a vascular source. In some embodiments, the cells may be a multilayered cardiac tissue construct. In some embodiments, the scaffold may be a single layer containing both the microchannels and the cells, wherein the cells are placed at a distance no more than 200 μm from a nutrient source, and wherein the thickness of the layer may be a thickness of from about 100 μm to about 600 μm. In some embodiments, the layer may be a thickness of from 200 μm to 400 μm. In some embodiments the layer may be a thickness of about 200 μm.

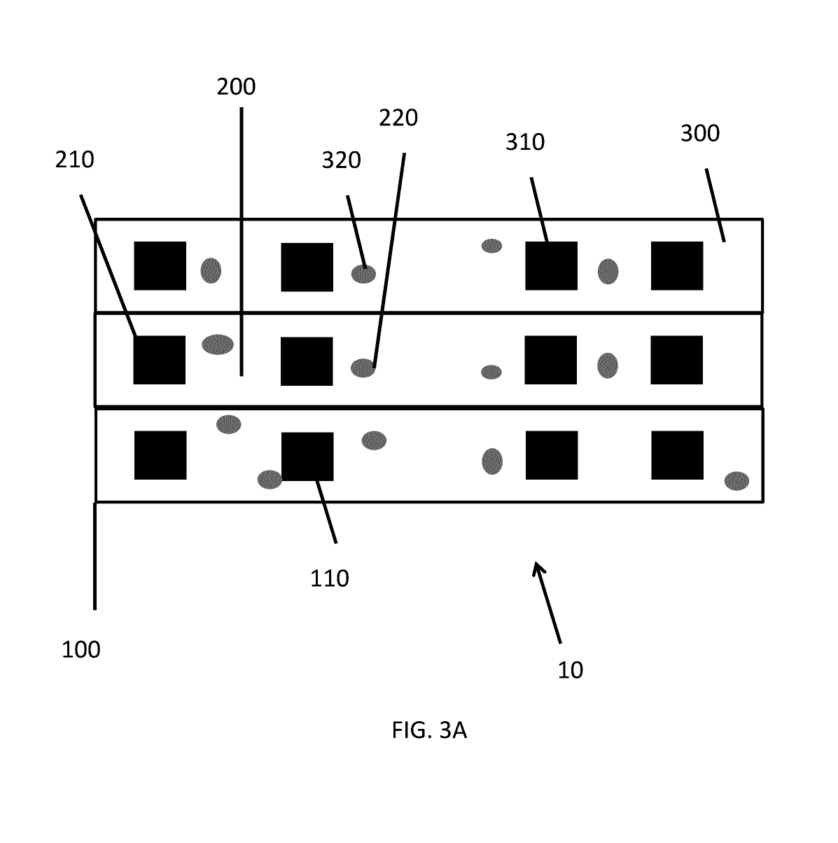

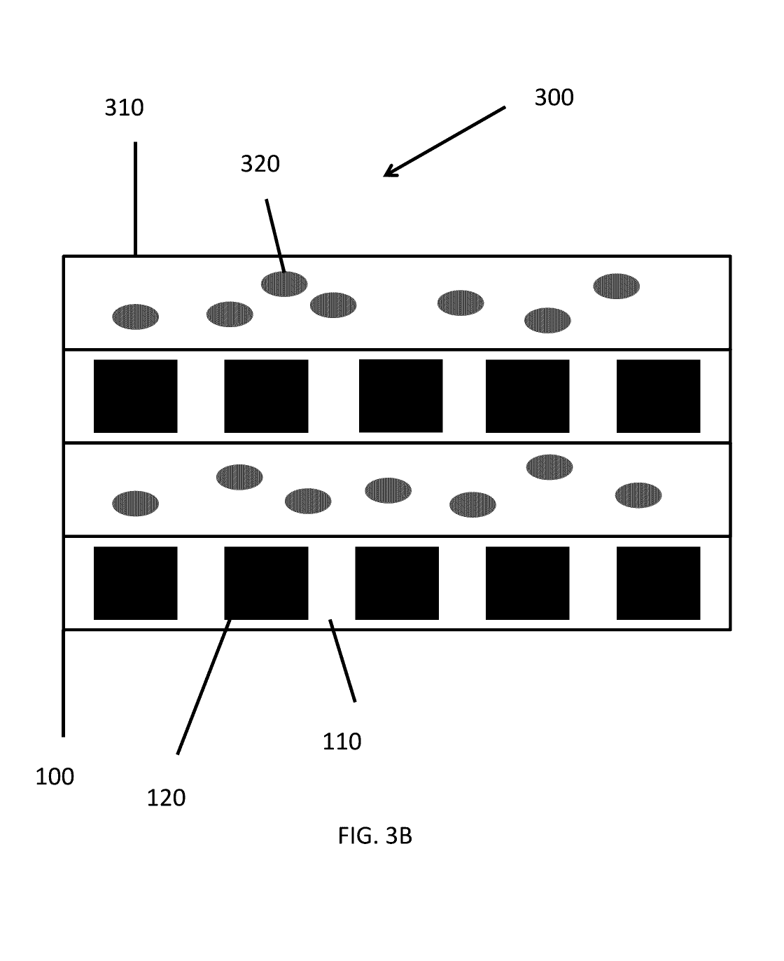

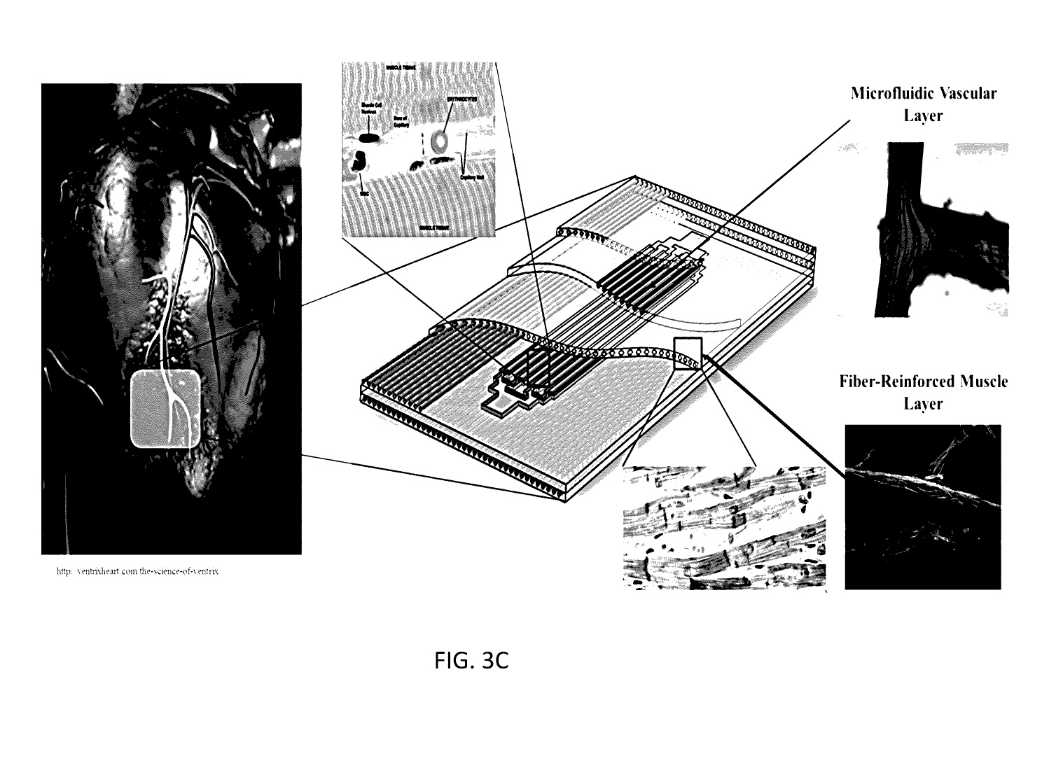

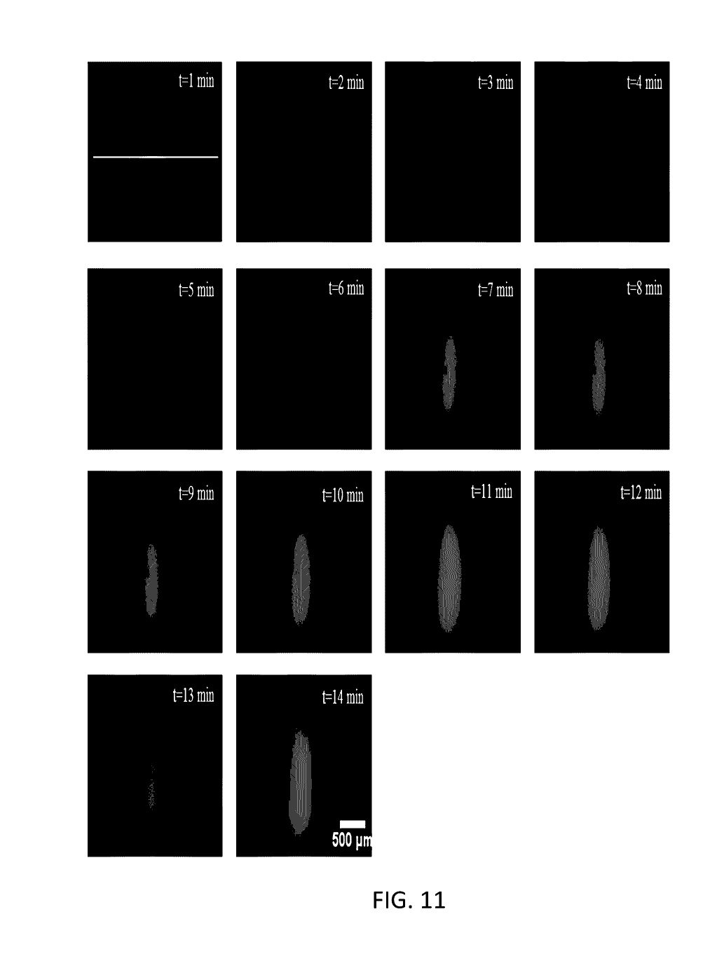

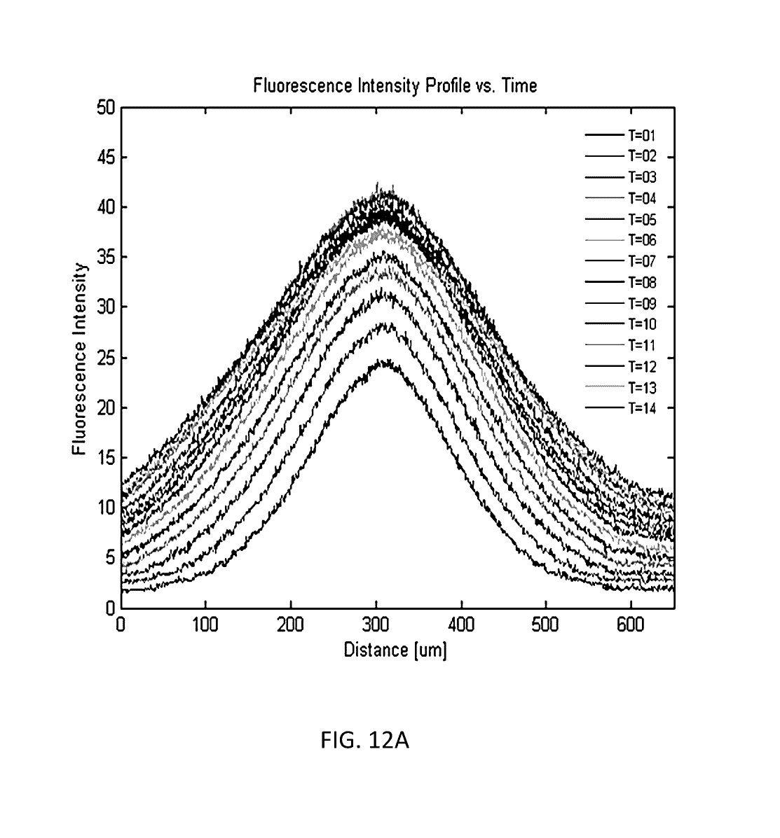

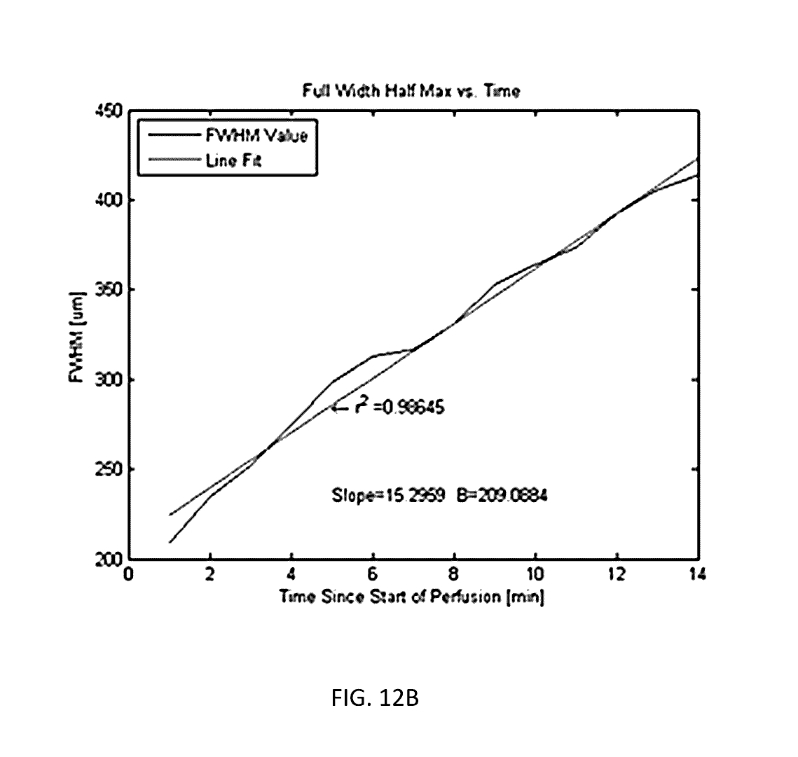

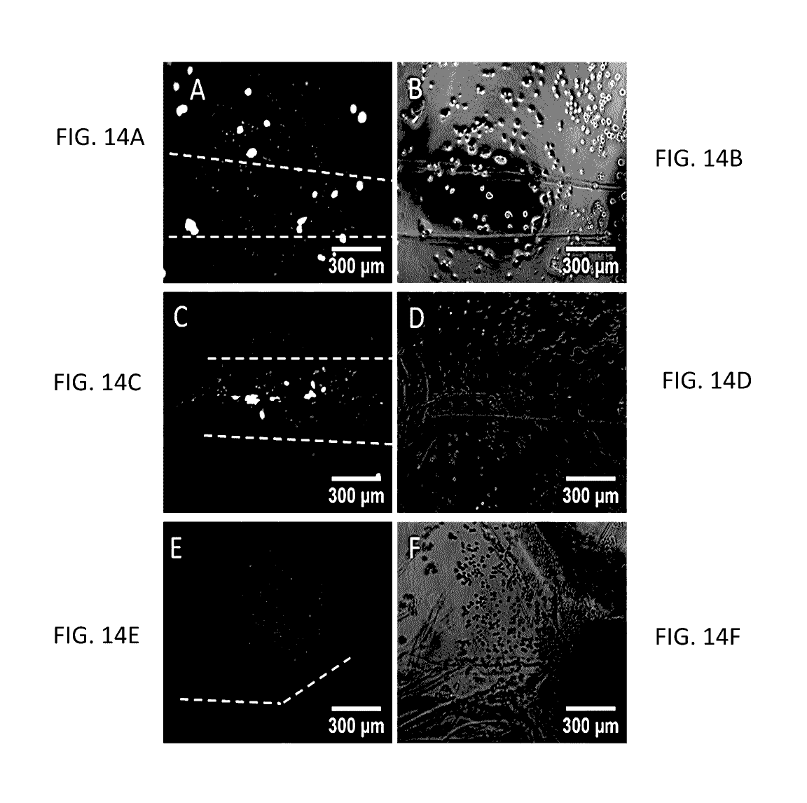

In some embodiments, as seen in FIG. 3A, a first microvascular layer 100 may be combined or stacked with one or more additional microvascular layers 200, 300 to provide a scaffold of multiple layers, wherein each layer may contain microchannels 110, 210, 310 and cells 120, 220, 320. In some embodiments the cells 120, 220, 320 may be seeded within the microvascular layer 100, 200, 300 during construction of the microvascular layer 100, 200, 300. In some embodiments, the cells may be endothelial cells, and the endothelial cells may be seeded within lumens of the microchannels (110, 210, 310) to yield an endothelialized microvascular network. In some embodiments, a first microvascular layer of the scaffold includes the microchannels and a second functional tissue layer includes the cells, the two layers defining a microvascular network, and the pattern may be repeated to form a multi-layer scaffold. In some embodiments, the scaffold may comprise multiple microvascular networks which may be stacked or layered. In some embodiments, the scaffold may be configured to promote the survival of endothelial cells or myocytes in a luminal or bulk gel fashion, or in some embodiments the cells may be cultured on the surface of the microvascular network. The enhanced survival may by via apposition of the nutrient source (in the microchannels) with the cells (e.g., myocytes such as neonatal rat ventricular cardiomyocytes or iPS-derived cardiomyocytes) and/or a functional tissue layer (e.g., engineered myocardium). Oxygen and nutrients may diffuse though the hydrogel as demonstrated in FIG. 11, FIG. 12 and FIG. 14. In some embodiments, the layer by layer construction may bring the cells (i.e., myocytes) to within 200 microns of a vascular source. Additionally, the microvascular network may facilitate the diffusion of nutrients, small molecules and oxygen from a perfusate contained in the microchannels to any surrounding tissue in a body after the scaffold is implanted. In some embodiments, the scaffold is perfusable, manufacturable and maneuverable. In some embodiments, the perfusate or nutrient source directed into and/or through the microchannels may contain growth factors, cytokines, nutrients or any other molecule that may be necessary or desirable for sustaining cell survival and/or growth. Additionally, it is understood that the microchannels may be used to deliver a drug or medication to the cells or to the location of implant should the scaffold be implanted.

Adaptation of this thin-film scaffold could be utilized to vascularize layer-by-layer constructs for regeneration of other tissues as well, such as skeletal muscle, skin, hepatic tissue or other engineered tissues that currently face similar vascularization problems to cardiac tissue engineering. Additionally, the single channel system can also be endothelialized. Such system could be used as a blood vessel model system for modeling angiogenesis, diapedesis of neutrophils.

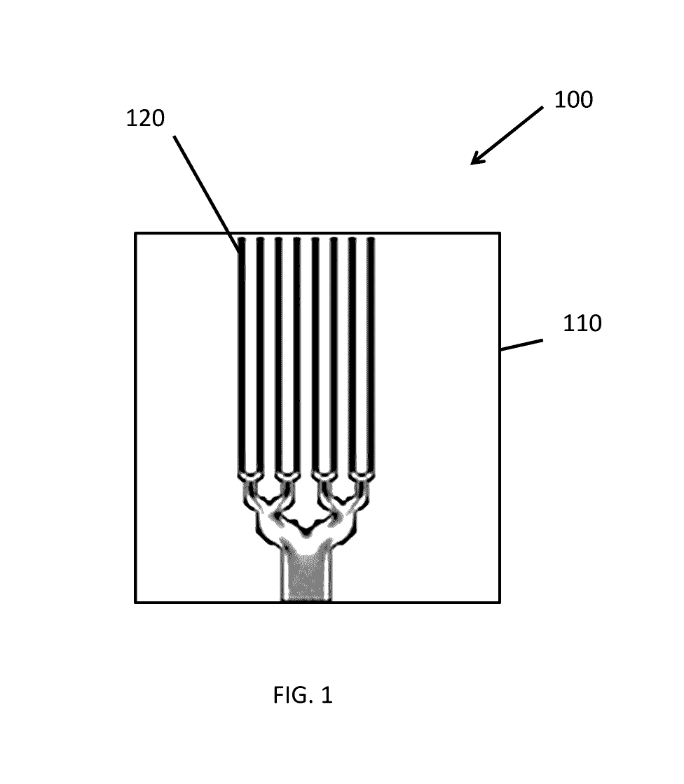

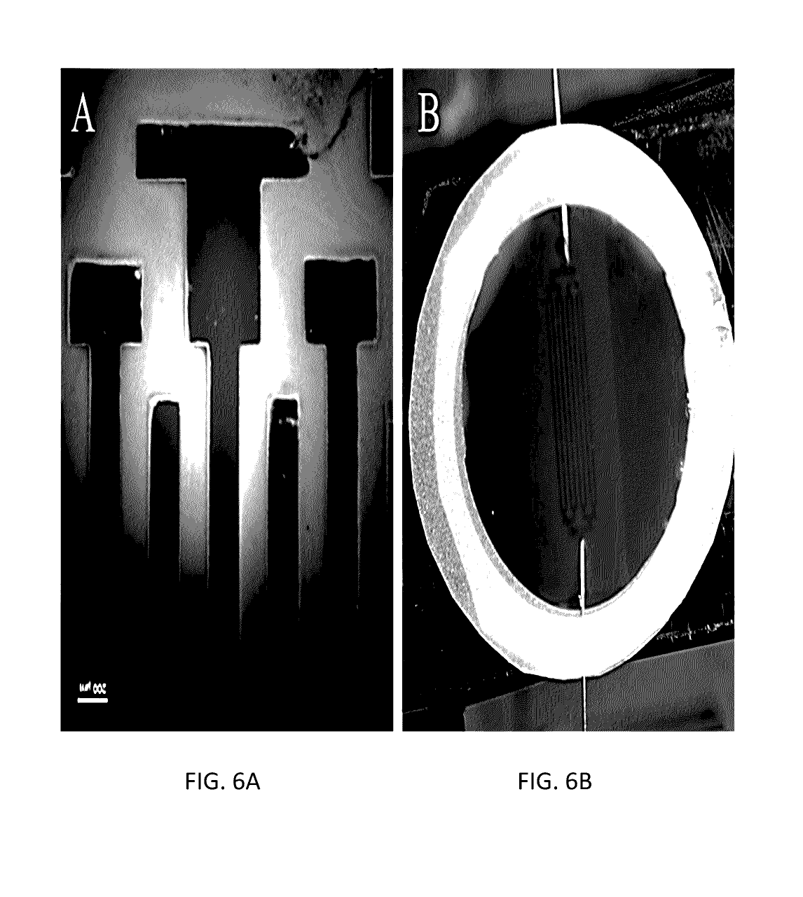

In reference to FIG. 1, in some aspects, the present disclosure provides a thin-film microvascular layer 100 formed of a thin film hydrogel 110 with an embedded microchannel network 120 capable of supporting continuous perfusion, the layer 100 configured to support a layer-by-layer construction of a vascularized tissue engineering construct or scaffold. In some embodiments the hydrogel comprises a polymerizable material. During the polymerization of the polymerizable material, long, interwoven, yet randomly oriented nano fibers may be created. In some embodiments of the microvascular layer 120, small particles may diffuse through the inter fibrillar spaces, while larger particles are significantly slowed by a microvascular layer meshwork. Small molecules such as O2, glucose and FITC may move through the hydrogel network 120 via a passive diffusion mechanism. Larger mesoscale components, such as 1 micrometer diameter spheres and mammalian cells, are constrained to the lumen of microvascular network (see FIG. 6) by the meshwork in the hydrogel.

In some embodiments, the microvascular network 120 may be endothelialized, wherein endothelial cells may be introduced into the lumens of the channels (not pictured) in the vascular network 120 to create a network of endothelialized channels, or channels that are coated with endothelial cells.

In some embodiments, the thickness of the thin film hydrogel 110 may be less than about 300 μm. In some embodiments, the thickness of the thin film 110 may be less than about 200 μm. In some embodiments, the thickness of the thin film 110 may be between about 100 μm and about 200 μm.

In some embodiments, the polymerizable material may be fibrin. In some embodiments, the thin film hydrogel 110 may be formed from other biocompatible materials used in the art for scaffold manufacturing, or may be formed with biocompatible materials in addition to fibrin. Low density materials are ideally suited to serve as the material for the scaffold due to their fibrillar nature, which, as discussed above, permits rapid diffusion of small particles and molecules through the gel. Fibrin, for example, is fairly simple to polymerize and is an endogenous protein (found in blood and clotting cascade). Additionally, it is the first scaffold deposited by the body in wound repair (thus, it is known to be a good cellular scaffold) and studies have shown it to have naturally angiogenic properties.

A challenge in using any low density material, like fibrin, for creating a scaffold that will support a microchannel network, is determining the correct density of the scaffold material. Hypodense (low density) gels, while having very favorable diffusion properties, may not maintain channel geometry. Hyperdense (high density) gels, in contrast, may be more mechanically stable, but it is known that more dense materials can have lower diffusion rates. In some embodiments, the final density of the thin film hydrogel 110 may be about 20 mg/ml. In some embodiments, the density of the thin film hydrogel 110 may be about 30 mg/ml. In some embodiments, the density of the thin film hydrogel 110 may be defined as a minimal density required to retain the shape of the channels 120. In some embodiments, the density of the thin film hydrogel 110 may be from about 10 mg/ml to about 40 mg/ml. In some embodiments, the density of the thin film hydrogel 110 may be from about 10 mg/ml to about 30 mg/ml. In some embodiments, the density of the thin film hydrogel 110 may be from about 10 mg/ml to about 20 mg/ml. In some embodiments, the density of the thin film hydrogel 110 may be from about 20 mg/ml to about 30 mg/ml.

In some embodiments, the hydrogel 110 may comprise fibrinogen, CaCl₂ and thrombin, wherein the fibrinogen is cleaved to fibrin during the polymerization process, leaving a minimal amount of uncleaved fibrinogen in the matrix.

In some embodiments, other materials may be used to form the thin film hydrogel 110, such as, for example, collagen, fibrinogen, laminin, hyaluronic add, agarose, alginate and combinations thereof, as well as synthetic polymers such as polyethylene glycol (PEG) and other synthetic materials, as well as combinations of synthetic scaffold materials and combinations of synthetic and natural materials, such as PEG and fibrin, may be used for the scaffolds.

Another challenge is sealing the layers together to stop leakage of flow between or within layers. In some embodiments, it can be achieved by differential cross linking of the hydrogels. For example, the flat, bottom layer can be cast before casting the top half of the microfluidic layer. The time difference will depend to the time it takes for the material to polymerize. Then, the top half of the network may be allowed to polymerize for a desired time, before moving it onto the bottom half. This may allow for some polymerization of the hydrogel to become intertwined with the mesh in the bottom layer, thus cross linking the two layers together. For example, for a material that takes about 10-15 minutes to totally polymerize, the time difference for the bottom layer may be approximately 15 minutes and for the top layer about 10 minutes.

Further, fabrication of a layer in a mold may be difficult, as the material (e.g., fibrin) may stick to the molds and tear upon removal. To overcome the challenge of sticking, the molds may be coated in an agent that inhibits sticking (e.g. bovine serum albumin (BSA)). Removal from the molds may be enhanced, with sticking further inhibited and crosslinking prevented from completing by removing the molded thin-film layer under a liquid bath (e.g., phosphate buffered saline (PBS)).

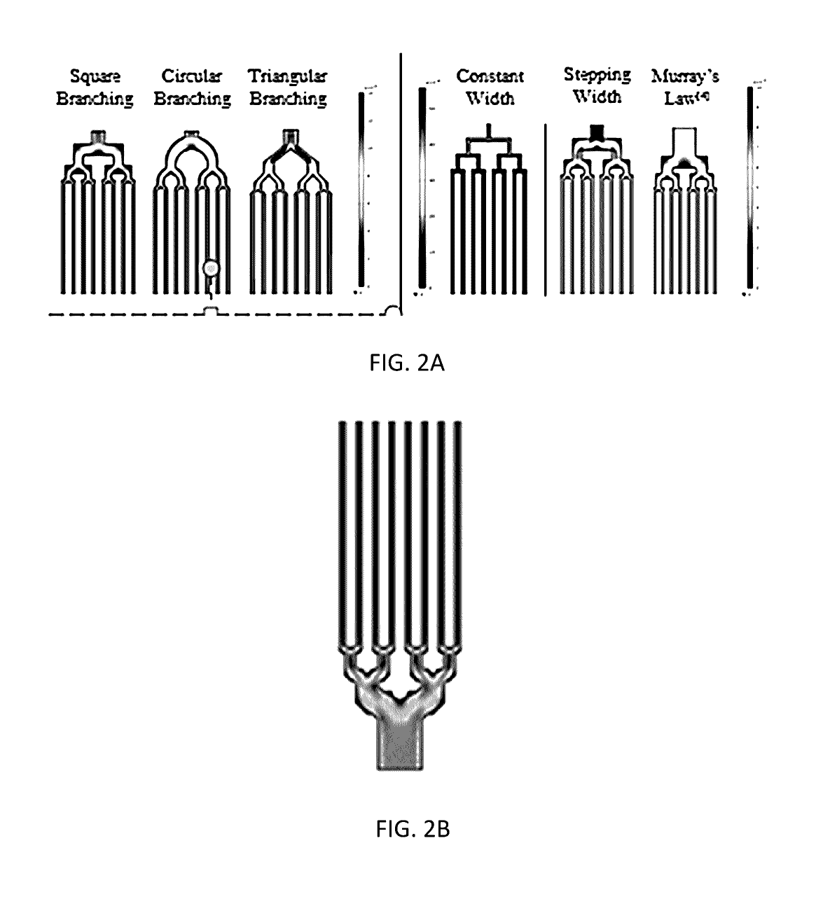

The microchannel network 120 may have various configurations. Referencing FIG. 2 illustrates non-limiting examples of suitable architectures of the microvascular network 120. In some embodiments, the network 120 may comprise a single channel. Additionally, the microvascular network 120 can be fabricated with a variety of additional dimensions, for example as seen in FIG. 2A, the microchannels may be branched. In some embodiments, the branching may be square, circular or triangular. In some embodiments, a width between the vessels may be constant, or may be determined by a stepping width function or via Murray's laws. The channel widths may range from 50 to 500 microns, or in some embodiments the channel widths may be from 10 to 800 microns, as may be necessitated by the design of the microvascular network. In some embodiments, the network 120 may be perfusable at a rate of at least 300 μm/sec. In some embodiments, the rate may be maintained using a syringe pump or other manual device such as a peristaltic pump, wherein the pump may connect to the scaffold via a needle, as seen in FIG. 6. In vivo, the flow rate will be entirely driven by cardiovascular hemodynamics, and as such, would depend upon the heart's cardiac output and the regulation of the microvascular flow.

In some embodiments, the network 120 is designed to maintain a constant rate of perfusion, without stagnation points. In some embodiments, a pulsatile perfusion may be maintained using a simple pulsatile flow apparatus.

The spacing between the channels can be altered, as well, though depending upon the material used, there is likely to be a minimum wall thickness, limited by the ability of the wall to support its own weight and moment. The thickness of the channels is limited by the photolithography techniques, it is therefore limited to a range of 1 to 400 um (based upon the limitation of the photolithography techniques). In some embodiments, as seen in FIG. 2B, the channels may have a hybrid or dynamic branching geometry using the best characteristics of each branching and width characteristics to generate a microchannel network 120 using advanced lithography techniques having a flow with minimal stagnation and a relatively uniform velocity.

In reference to FIG. 3A, in some embodiments, the present disclosure provides a scaffold 10 comprising one or more microvascular layers 100, 200, 300. Each layer 100, 200, 300 may further comprise a microchannel network 110, 210, 310 and cells 120, 220, 320. The layers 100, 200, 300 may be configured to sustain growth or survival of the cells 120, 220, 320 disposed in that layer or neighboring layers 100, 200, 300. In some embodiments, the cells 120, 220, 320 may be disposed at a distance from the microchannels 110, 210, 310 such that nutrients may diffuse from the microchannels 110, 210, 310 to the cells 120, 220, 320. The layers may be prepared separately and combined together, or the scaffold can be prepared as a single layer with increased thickness to support one or more microchannel networks at different levels inside the scaffold. In some embodiments, the thickness of the layer is such that the cells are always less than 200 μm from the vasculature or nutrient source. In embodiments where the cells 120, 220, 320 are seeded in the microvascular layers 100, 200, 300, the spacing between the channels 110, 210, 310, as well as the amount of fibrin above the channels 110, 210, 310 in the microvascular layer 100, 200, 300, may be used to determine the maximum thickness of the microvascular layer 100, 200, 300. In some embodiments, the thickness and shaping may be determined mathematically (e.g., via trigonometric and geometric calculations), alternatively, in some embodiments a scale model may be drawn using circles with radius of no more than 200 microns at all of the corners, wherein the microchannels 110, 210, 310 may be positioned so that any diffusion circles no longer overlap.

The layers 100, 200, 300 may be stacked in a layer by layer construction. In some embodiments, the layers 100, 200, 300 may be endothelialized. In reference to FIG. 3B, in some embodiments, the present disclosure provides a scaffold construct 300 that may include one or more thin-film microvascular layers 100 comprising the microchannels 120, alternating with one or more functional tissue layers 310. The layers may be prepared separately and combined together, or the scaffold can be prepared as a single multilayer scaffold with increased intralayer thickness in some embodiments to support one or more microchannel networks at different levels inside the scaffold and within each layer. The tissue scaffolding layer 310 may be seeded with one or more cell types 320, depending on the tissue being engineered. The first 100 and second layer 310 comprise a perfused microvascular network, wherein the microvascular layer 100 is configured to deliver or supply nutrients to cells 320 which may be preseeded in the functional layer 310. In some embodiments, the cells 320 may be seeded in such a manner so that they are disposed no more than 200 microns from a microchannel 120 or nutrient source. In some embodiments, the cells 320 in the functional tissue layer 310 may be fed by either or both of the microvascular layers located above (not pictured) and below 100 the functional tissue layer 310. The nutrients may diffuse from the microvascular layer 100 to the functional layer 310. In some embodiments, the scaffold 300 may comprise multiple perfused microvascular networks, which may be stacked or layered on top of one another just as the individual layers may be configured to be stacked or layered. In some embodiments, the cells 320 can be preseeded in both the microvasular layer 100 and the functional layer 310. In some embodiments, seeding the cells 320 in either layer 100, 310, in a bulk fashion, may be done by casting the gel with a suspension of the cells 320 (at the desired density) in place of the PBS.

In some embodiments, the scaffold 300 constructs of the present disclosure may be used to engineer cardiac tissue. In some embodiments, as shown in FIG. 3C, the scaffold 300 for regenerating cardiac tissue may comprise alternating layers of functional, contractile myocardial tissue and thin film layers with a discrete, intact, perfusable vasculature.

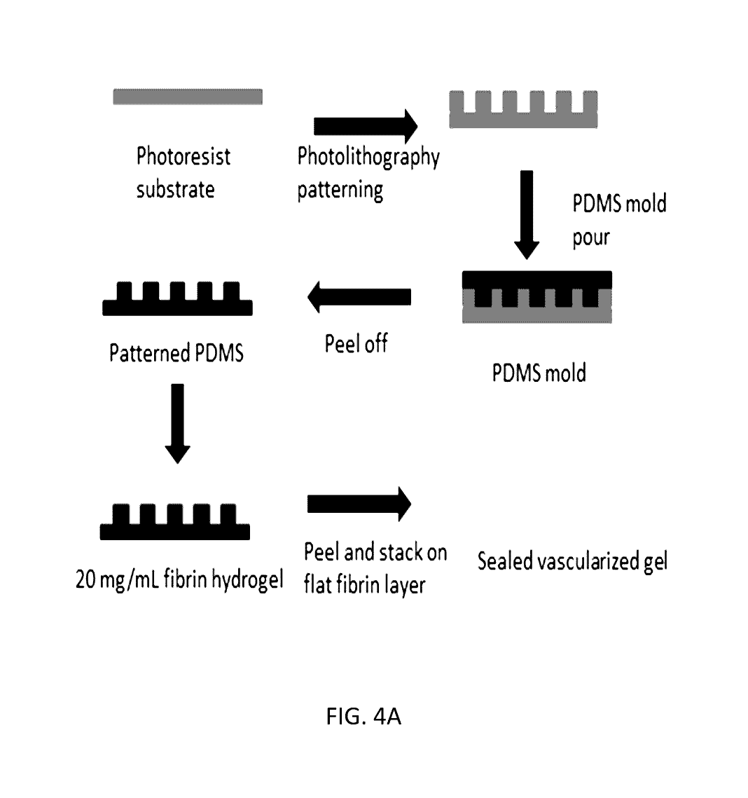

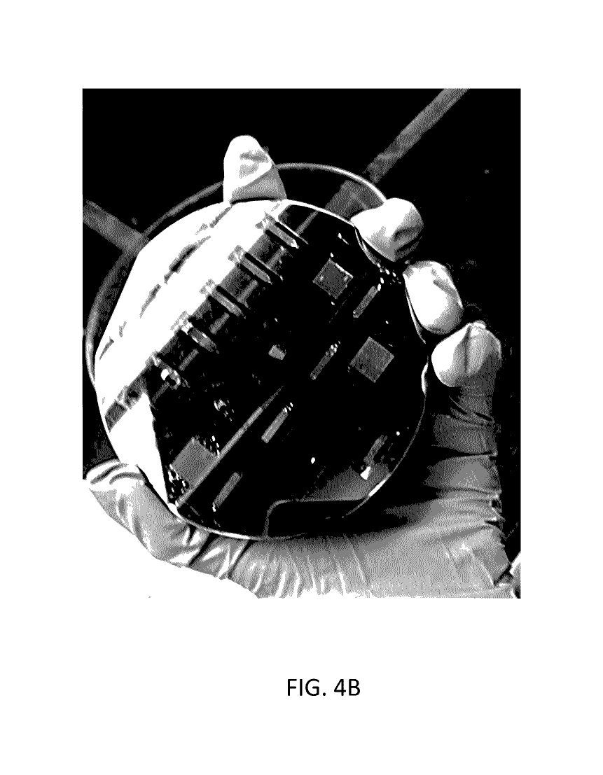

Various methods may be used to create the microchannel network 120 in the thin film hydrogel 110. In some embodiments, the thin film hydrogel 110 may be patterned with the network 120 using lithography. By way of a non-limiting example, as shown in FIG. 4, a design for a microvascular network may be printed onto a photomask, which may be used during a photolithography process to create a silicon wafer. Once the silicon wafer is fabricated, it may be prepared to be used as a mold for a biocompatible polymer such as an elastomeric polymer (e.g., polydimethylsiloxane (PDMS)). The material for the film may be added to the elastomeric PDMS and may be cured or crosslinked to form a film representing half of a layer comprising the microvascular network. A second half layer may be fabricated concomitantly to the first layer, and the layers may be combined to form a complete thin-film layer with an embedded microchannel network within the layer. The layers may then be seeded with cells on top of the layers or in a lumen of the channels, or alternatively, as mentioned previously, the layers containing microchannels may be stacked with layers preseeded and containing cells to form diffusible microchannel networks. In some embodiments, the microchannel network is created using a scaffold mask, the scaffold mask being prepared from the silicon wafer or photomask. The scaffold mask may be reusable. In some embodiments, a first scaffold mask is used to make a first half layer, and a second scaffold mask is used to make a second half layer. In some embodiments, the second layer may be fabricated after the first half layer, provided that any required modification to either half layer, such as crosslinking (e.g. of fibrin) is not performed until both half layers are ready to be combined to form a final layer or microvascular network.

The methods and materials of the present disclosure are described in the following Examples, which are set forth to aid in the understanding of the disclosure, and should not be construed to limit in any way the scope of the disclosure as defined in the claims which follow thereafter. The following examples are put forth so as to provide those of ordinary skill in the art with a complete disclosure and description of how to make and use the embodiments of the present disclosure, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

### EXAMPLES

### Engineered Fibrin Vascular Network

In reference to FIG. 4A, schematic of design of fibrin-based microfluidic network is presented. A 4 inch diameter silicon wafer (University Wafers, Boston) was dehydrated on a hot plate for 5 minutes, before being spin-coated with SU-8 2035 (MicroChem, Westborough) at a speed of 1250 rpm. The coated wafer was then pre-baked at 65° C. for 5 minutes, before being baked for 15 minutes at 95° C. The photomask was then placed atop the wafer and the combination was exposed to 365 nm UV light for 14 seconds at an intensity of 23.4 mW/cm². The wafer was then baked at 65° C. for 5 minutes, 95° C. for 9 minutes, and finally 65° C. for 3 more minutes. Next, the photoresist was developed in the developer solution with gentle agitation for approximately 8 minutes. The resultant wafer can be seen in FIG. 4B

Once the silicon wafer was fabricated, it was prepared to be used as a mold for the elastomeric polymer PDMS. The first step was the fluorination of the surface, in which the silicon surface was treated with Tridecafluoro-1,1,2,2-tetrahydrooctyl)trichlorosilane (TFOCS), which binds to the surface, making it hydrophobic. To coat the surface with TFOCS, the wafer was placed in a vacuum chamber with 40 μL of TFOCS for 1 hour. 110 grams of SYLGARD 184 (Dow Corning, Midland) was created by mixing 100 g of elastomer base with 10 g of the elastomer curing agent. This mixture was poured over the TFOCS-treated wafer and cured in an oven at 65° C. for four hours. The central region of PDMS, the region above the wafer, was then excised using a scalpel and 49.5 g of PDMS (45 g base, 4.5 g curing agent) was added to the void and cured, such that no dust would settle on the wafer.

Using photolithography, 100 μm tall channels were printed onto a silicon wafer and cast into PDMS. A fibrin hydrogel was then created by mixing 670 μL fibrinogen (30 mg/mL, MPBiomedicals, Santa Ana), 150 μL thrombin (2.35 U/mL, Sigma Aldrich, St. Louis), 100 μL CaCl2 (40 mM, EM Science) and 80 μL PBS (VWR, Bridegport) or 80 μL cell suspension. 150 μL of the solution was cast into ¾″ vellum film rings placed onto the BSA-coated PDMS molds.

Fibrin gels may be cross-linked on the benchtop, before being submerged in DI water or 1×PBS. At this point, the vellum paper ring containing the imprinted fibrin gel may be carefully removed from the PDMS mold, ensuring that the gel remained submerged the entire time. Gels may then be placed on PDMS coated glass slides and imaged for quality assurance.

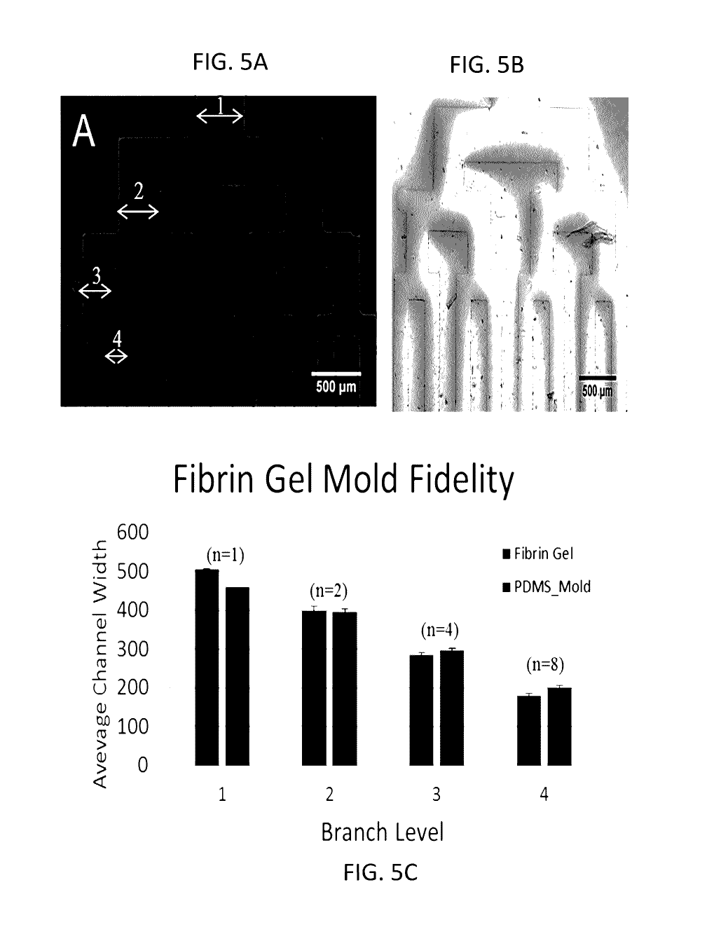

FIG. 5 verifies branching dimensions in PDMS (5A) and fibrin (5B) molds. FIG. 5C illustrates a quantitative comparison of the channel widths in fibrin and PDMS. ImageJ (NIH, Bethesda) was used to measure the width dimensions in fibrin and PDMS to verify pattern retention. FIG. 5C displays a comparison of PDMS and fibrin for branching geometries. The expected and actual widths, while statistically different at the third and fourth branch level are very similar, with a small standard deviation.

Microfluidic Perfusion

FIGS. 6A and 6B illustrate a micro-bead loaded microvascular network. FIG. 6A illustrates a discretely loaded fibrin microvascular network perfused with blue microbeads. FIG. 6B illustrates a layered fibrin μVN perfusion with blue latex microspheres (Polysciences, Inc., Warrington) perfused into the microvascular network using syringe pump.

Hydrogels were loaded with 1 μm diameter blue latex micro-beads (Polysciences, Inc., Warrington, Pa.) using a drip-perfusion method. FIG. 6A shows channels filled with micro particles, evidence that these microvascular devices can be discretely loaded without significant leakage from channels.

Single Channel Perfusion

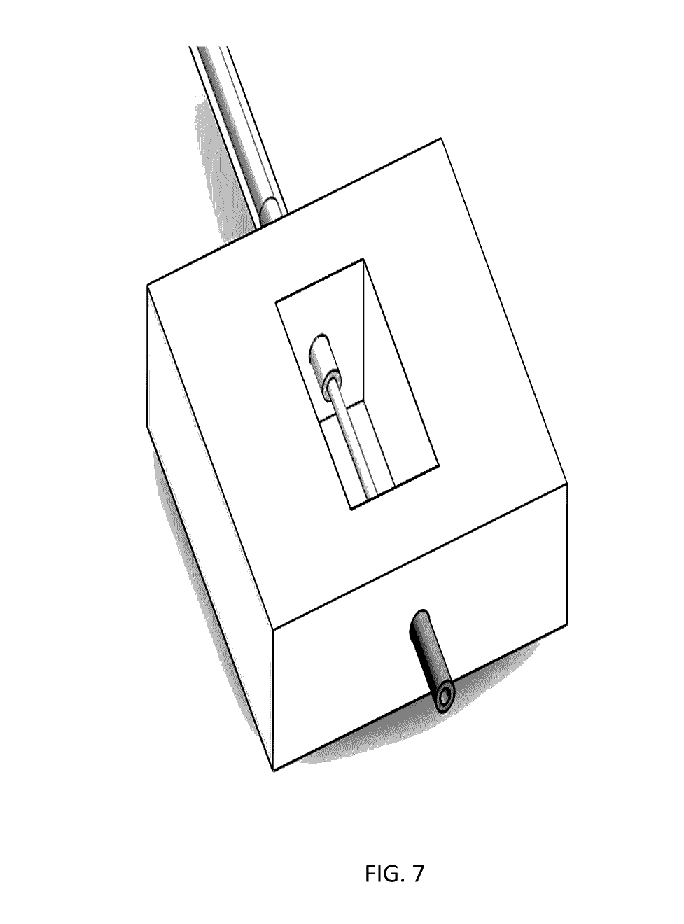

In reference to FIG. 7, the principles of continuous perfusion in an engineered fibrin vascular system are demonstrated using a single channel fibrin hydrogel was created. This system utilizes a 30 gauge needle, nested within 23G stubs. Removal of the 30G needle generated a 300 μm diameter channel which can be perfused by attaching PE-50 tubing to the 23G stubs. These models can withstand high changes in volumetric flow rate and provide a useful model to study diffusion of molecules and viability of cells cultured on the surface of the gel.

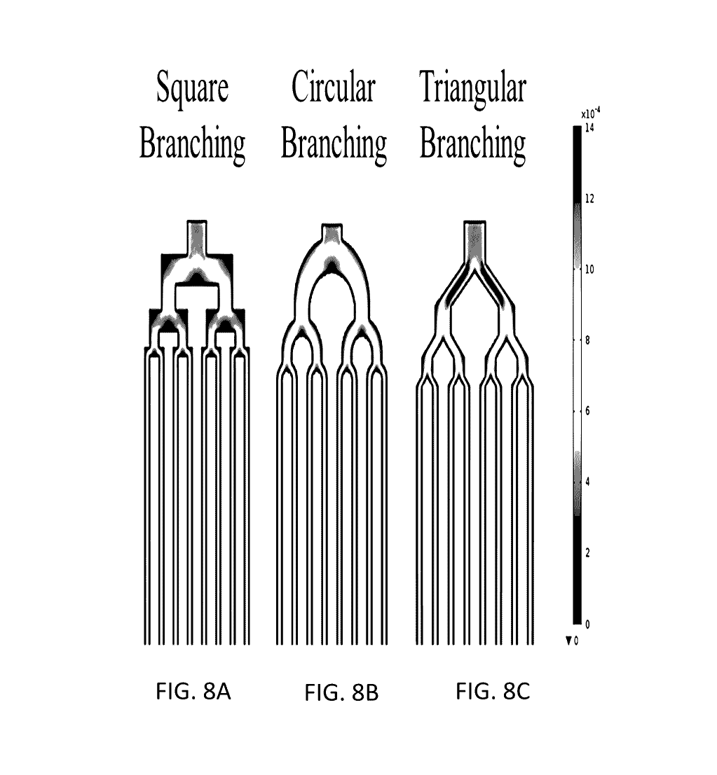

Microfluidic Design Computational Fluid Dynamics (CFD, COMSOL, Burlington) was used to analyze the velocity and pressure profiles (not shown) of various channel widths and junction geometries. Each system was modeled with a 300 μm/sec flow velocity in the diffusion channels. As shown in FIGS. 8A-C, three junction geometries were tested: square (FIG. 8A), circular (FIG. 8B) and triangular branching (FIG. 8C). Qualitative analysis suggested that circular branching provided the most uniform flow velocities while triangular channels minimized flow dead zones in the corners and adjacent to inlets and branch points. The pressure needed to drive the square branching junctions was the lowest.

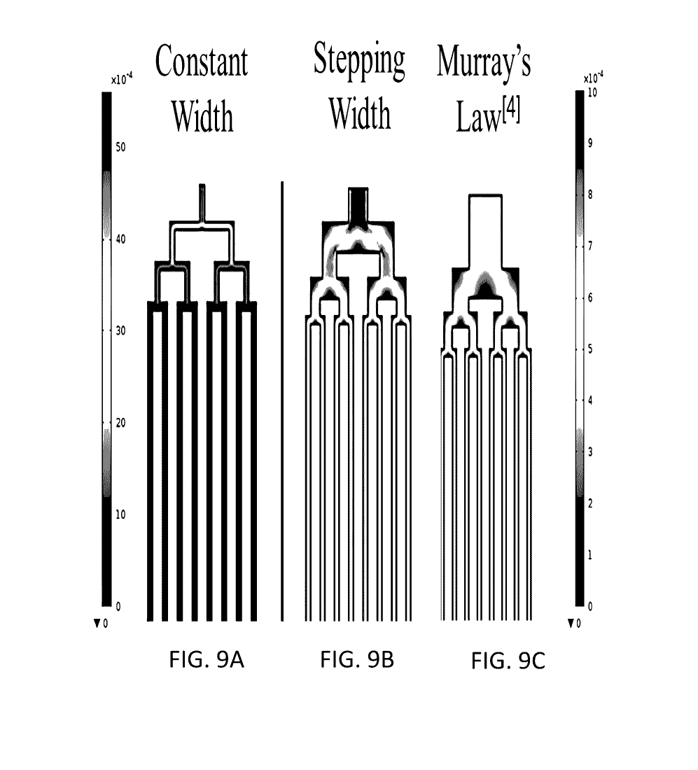

FIGS. 9A-C illustrates a width profile analysis of the vascular network. Three width profiles were tested: Constant Width (9A), and Stepping Width (9B) (decreasing width of 100 μm/branch) and Murray's Law (9C). Murray's Law showed the most uniform flow velocity of the three. The stepping width showed similar pressure drops and uniformity of flow, though not to the same degree of uniformity seen in the Murray's Law model.

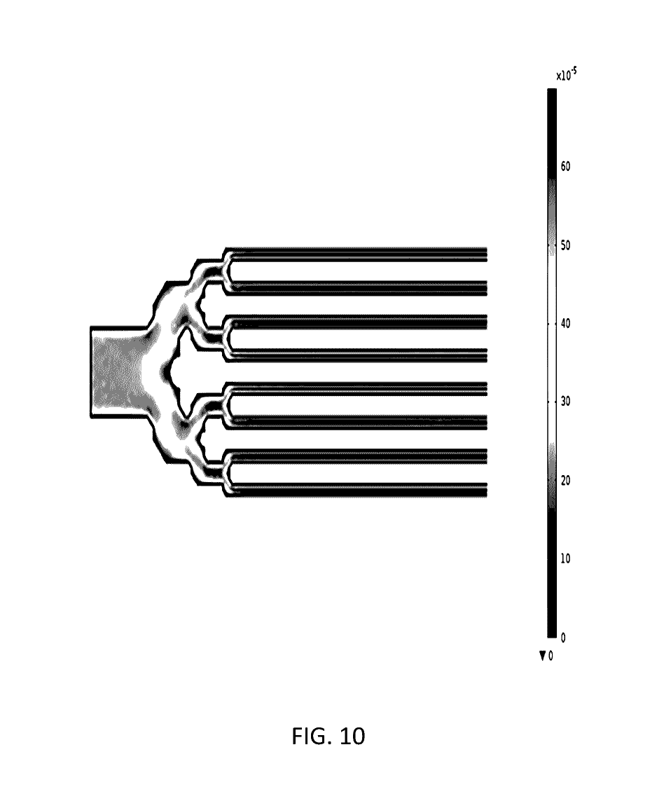

FIG. 10 illustrates another potential embodiment of the microfluidic network, which may minimize driving pressure, number of dead zones, and showed a highly uniform velocity profile. This is a hybrid design conceived from the best aspects of each model described above.

FITC Diffusion Study

FIG. 11 illustrates graphs of flow and diffusion of FITC in a single channel fibrin channel over time, with the top left image taken at 1 minute and the bottom right image taken at 14 minutes. In order to examine the diffusion of small molecules from the channels, a single channel was perfused with FITC for 14 minutes at a flow rate of 82 μL/hr, which was calculated assuming a 300 μm/sec flow velocity, a common flow rate through capillaries in humans. Qualitatively, initial channel localization of the FITC was observed, though the molecules were observed to diffuse outwards through the gel at a fairly constant diffusion rate starting immediately after initial channel perfusion.

Perfusion-Mediated Cell Viability and Survival

FIG. 12 illustrates results of an analysis of fluorescence intensity versus change in distance from the center of the channel, (centered at approximately 300 μm in this plot). In FIG. 12A, fluorescence intensity profile is measured along the length of a line which is shown in the top-left image of FIG. 11. This distribution indicates uniform and symmetric diffusion of FITC through the gel, on each side of the channel. Each time point shows an increase in intensity (indicated by the upwards shift of the curve), and an increase in diffusion distance (indicated by increasing distribution width). In FIG. 12B, a graph of Full Width at Half Maximum (FWHM) distance versus time is shown, illustrating that the relationship between the FWHM of the intensity profile and time is linear. A linear regression indicated that the FWHM increased nearly constantly at a rate of 15.29 μm/sec. As FITC is nearly twice the size of glucose, and many times larger than 02 it is suggested that these two molecules should pass through the gel at an even faster rate than FITC.

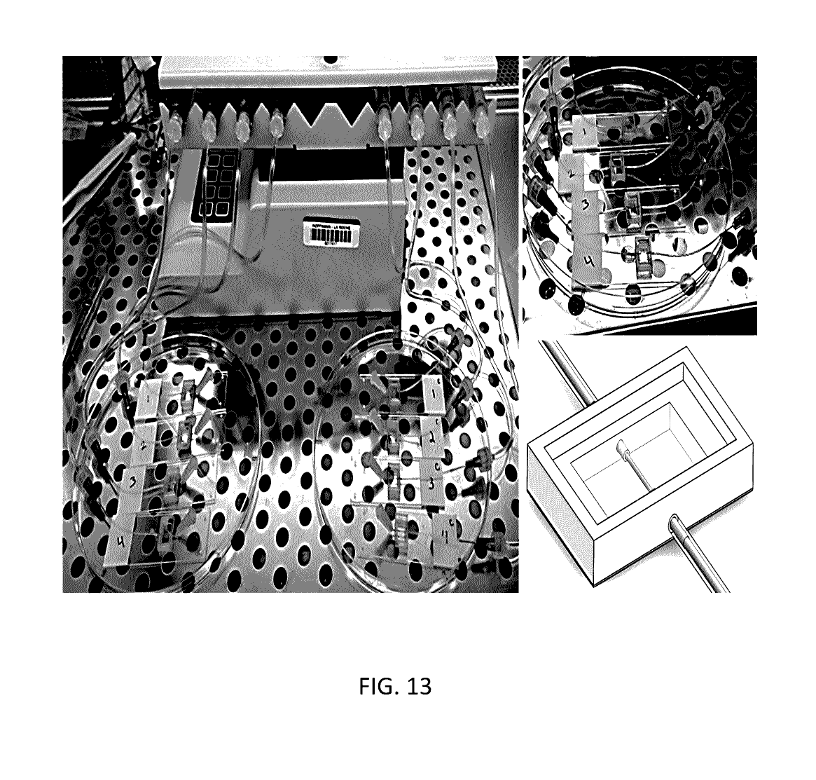

FIG. 13 illustrates a Cellular Viability Testing Apparatus. 100,000 C₂C₁₂ cells, cultured at 37° C. and 5% CO2 with a C₂C₁₂ modified DMEM nutrient source, were seeded on top of each single channel system. After allowing the cells to attach for four hours, the medium was replaced with PBS, then cell culture medium or PBS was perfused through the single channel for 24 hours at a flow rate of 82 μL/hr. To verify the diffusion of medium through the gel, the cells were stained with CFDA and ethidium bromide to distinguish the live and dead cells.

FIG. 14 illustrates fluorescent microscopy images of cellular viability study at 24 hours. Perfusion of the single channel system displayed living cells in the medium perfused channel with virtually no living cells in the PBS perfused channel. FIG. 14A-D are images from gels perfused with media, while FIGS. 14E and 14F are from a gel perfused with PBS. These results suggest that cellular viability was greater on the medium perfused scaffolds than it was on the PBS perfused scaffolds. Although not all cells survived, the clear difference between the experimental and control hydrogels suggests gels that media perfused gels increased cellular viability.

In summary, a thin, microengineered fibrin vascular network was created. It showed: high pattern fidelity and the ability to localize fluid within the engineered channels. It was shown that the microfluidic network can be optimized, generating a system with a lower overall resistance, fewer flow dead zones and a more uniform velocity than each of the component systems tested. The network can have high rates of diffusion for small molecules and ultimately increases cellular survival when used as a mechanism for medium delivery

All patents, patent applications, and published references cited herein are hereby incorporated by reference in their entirety. It should be emphasized that the above-described embodiments of the present disclosure are merely possible examples of implementations, merely set forth for a clear understanding of the principles of the disclosure. Many variations and modifications may be made to the above-described embodiment(s) without departing substantially from the spirit and principles of the disclosure. It will be appreciated that several of the above-disclosed and other features and functions, or alternatives thereof, may be desirably combined into many other different systems or applications. All such modifications and variations are intended to be included herein within the scope of this disclosure, as fall within the scope of the appended claims.

What is claimed is:

1. A scaffold comprising at least one microvascular layer formed from a thin-film fibrin, the microvascular layer configured to sustain and promote growth of cells and having one or more microfluidics channels embedded in the microvascular layer, the channels configured to contain nutrients needed for growth of the cells, and the channels configured to permit diffusion of the nutrients from the channels to the cells.

2. The scaffold of claim 1, wherein the microvascular layer comprises a thickness of from about 100 microns to about 600 microns.

3. The scaffold of claim 1, wherein the cells are disposed in the microvascular layer.

4. The scaffold of claim 1, wherein the cells are disposed in the microvascular layer at a distance of no greater than 200 microns from the microfluidics channels.

5. The scaffold of claim 1, wherein the scaffold comprises multiple microvascular layers, the layers being stacked on top of each other.

6. The scaffold of claim 1 further comprising at least one functional tissue layer having cells disposed within the functional layer, the microvascular layer being configured to permit diffusion of the nutrients from the microvascular layer to the cells in the functional layer.

7. The scaffold of claim 6, wherein the functional layer and the microvascular layer comprise a thickness of from about 100 microns to about 200 microns.

8. The scaffold of claim 6, wherein the scaffold comprises multiple functional layers and multiple microvascular layers, the layers being stacked and arranged such that at least one functional layer is positioned on each side of a microvascular layer.

9. The scaffold of claim 6, wherein the cells are disposed in the functional layer at a distance of no greater than 200 microns from the microfluidics channels in the microvascular layer.

10. The scaffold of claim 1, wherein the microfluidics channels comprise a width of from 10 to 800 microns, and a height of from 10 to 190 microns.

11. The scaffold of claim 1, wherein the cells are cardiac cells.

12. A method for sustaining cell survival in an individual comprising:
providing a scaffold, the scaffold comprising at least one microvascular layer formed from a thin-film fibrin and configured to sustain and promote growth of cells, the microvascular layer having one or more microfluidics channels embedded in the microvascular layer, the channels configured to contain nutrients needed for growth of the cells, and the channels configured to permit diffusion of the nutrients from the channels to the cells;
inoculating the microvascular layer with the cells; and
positioning the scaffold at a desired location within the individual.

13. The method of claim 12, wherein the scaffold comprises more than one microvascular layer, the scaffold being constructed in a layer by layer manner to achieve a layered scaffold of a desired thickness.

14. The method of claim 12, wherein the individual comprises an individual who has sufferered ischemic or reperfusion damage to a tissue.

15. The method of claim 12, wherein the scaffold further comprises a functional tissue layer having cells disposed within the functional layer, the microvascular layer being configured to permit diffusion of the nutrients from the microvascular layer to the functional layer, the microvascular and functional layers defining a perfused tissue scaffold.

16. A method of manufacturing a thin-film fibrin scaffold comprising:
filling a first scaffold mold with a fibrin hydrogel, the hydrogel having a concentration of from 10 to 30 mg/ml fibrin, and cross-linking the fibrin hydrogel to yield a first half layer of a thin-film fibrin scaffold;
concomitantly to filling the first mold, filling a second scaffold mold with the fibrin hydrogel, and cross-linking the fibrin hydrogel to yield a second half layer of the thin-film fibrin scaffold; and
combining the two half layers before the cross-linking is complete to yield a thin-film fibrin scaffold, the scaffold comprising one or more microfluidics channels, the channels configured to contain nutrients needed for growth of cells and configured to permit diffusion of the nutrients from the channels to the cells.

17. The method of claim 16, further comprising before filling the first scaffold mold:
patterning a mask for a scaffold mold on to a negative photoresist substrate to yield a patterned substrate;
pouring an organic compound into the patterned substrate and allowing the organic compound to harden, yielding a scaffold mold.

18. The method of claim 16, wherein the organic compound is PDMS.

19. The method of claim 16, wherein the patterning comprises photolithography.

20. The method of claim 16 further comprising seeding cells onto the scaffold.
